(19) United States
(12) Patent Application Publication
(10) Pub. No.: US 2014/0328789 A1
(43) Pub. Date: Nov. 6, 2014

(54) pH-RESPONSIVE CELL SCAFFOLD AND METHOD OF USING SAME

(71) Applicant: Research Foundation of the City University of New York, New York, NY (US)

(72) Inventors: Debra T. Auguste, Arlington, MA (US); Jin-Oh You, Cambridge, MA (US)

(21) Appl. No.: 14/269,467

(22) Filed: May 5, 2014

Related U.S. Application Data
(60) Provisional application No. 61/819,180, filed on May 3, 2013.

(51) Int. Cl. A61K 31/785; C12N 5/071

(52) U.S. Cl. CPC A61K 31/785; C12N 5/0602; C12N 2533/40
USPC 424/78.35; 435/396; 521/149

(57) ABSTRACT

A pH-responsive cell scaffold for growing a cell culture is disclosed. The cell scaffold has pores in which biological cells may be disposed. As the pH of the local environment drops, the cell scaffold swells to draw in additional oxygen and/or other nutrients. The increased supply of oxygen and/or nutrients increases the longevity of the cells. In some embodiments, the cell scaffold induces a change in gene expression that promotes wound healing.

### CROSS-REFERENCE TO RELATED APPLICATIONS

This application is a non-provisional of U.S. Patent Application Ser. No. 61/819,180 (filed May 3, 2013), the entirety of which is incorporated herein by reference.

### STATEMENT OF FEDERALLY SPONSORED RESEARCH OR DEVELOPMENT

This invention was made with Government support under contract number DMR-0820484 awarded by the National Science Foundation. The work was performed in part at the Center for Nanoscale Systems (CNS), a member of the National Nanotechnology Infrastructure Network (NNIN) which is supported by the National Science Foundation under award number ECS-0335765.

### BACKGROUND OF THE INVENTION

The subject matter disclosed herein relates to cell scaffolds for biological cells.

Porous, three-dimensional cell scaffolds are useful as tissue engineering scaffolds. The cell scaffold provides the structural support for cell attachment and survival. Given appropriate environmental conditions, scaffolds can support the growth of function tissue. However, post-implantation analysis shows that cells become paralyzed without a preformed vascular network. Limited diffusion of oxygen and nutrients from surrounding vessels results in permanent cell damage and death. Efforts to improve cell survival and vascularization have led to the fabrication of scaffolds with highly interconnected pores and fabricated vascular networks. None of these cell scaffolds has proven to be entirely satisfactory. An alternative cell scaffold is therefore desired.

The discussion above is merely provided for general background information and is not intended to be used as an aid in determining the scope of the claimed subject matter.

### BRIEF DESCRIPTION OF THE INVENTION

A pH-responsive cell scaffold for growing a cell culture is disclosed. The cell scaffold has pores in which biological cells may be disposed. As the pH of the local environment drops, the cell scaffold swells to draw in additional oxygen and/or other nutrients. The increased supply of oxygen and/or nutrients increases the longevity of the cells.

An advantage that may be realized in the practice of some disclosed embodiments of the cell scaffold is an increase in the longevity of the cells. In some disclosed embodiments, the cell scaffold induces a change in gene expression that promotes wound healing.

In a first embodiment, a pH-responsive cell scaffold is provided. The cell scaffold comprises a polymeric scaffold formed from a polymerization reaction of a reaction mixture comprising a pH-nonresponsive monomer and a pH-responsive monomer, the polymerization reaction forming a copolymer with a first pKa between about 5 and about 7.5. The cell scaffold comprises a plurality of pores, wherein the polymeric scaffold swells when exposed to an environment with a pH below the first pKa.

In a second embodiment, a method of growing a cell culture is provided. The method comprises steps of permitting biological cells to be disposed in a plurality of pores in a polymeric scaffold, the polymeric scaffold being formed from a polymerization reaction of a reaction mixture comprising a pH-nonresponsive monomer and a pH-responsive monomer, the polymerization reaction forming a copolymer with a first pKa between about 5 and about 7.5, wherein the polymeric scaffold swells when exposed to an environment with a pH below the first pKa. The method further comprises allowing the biological cells to adsorb nutrients from an ambient environment and grow a cell culture.

This brief description of the invention is intended only to provide a brief overview of subject matter disclosed herein according to one or more illustrative embodiments, and does not serve as a guide to interpreting the claims or to define or limit the scope of the invention, which is defined only by the appended claims. This brief description is provided to introduce an illustrative selection of concepts in a simplified form that are further described below in the detailed description. This brief description is not intended to identify key features or essential features of the claimed subject matter, nor is it intended to be used as an aid in determining the scope of the claimed subject matter. The claimed subject matter is not limited to implementations that solve any or all disadvantages noted in the background.

### BRIEF DESCRIPTION OF THE DRAWINGS

So that the manner in which the features of the invention can be understood, a detailed description of the invention may be had by reference to certain embodiments, some of which are illustrated in the accompanying drawings. It is to be noted, however, that the drawings illustrate only certain embodiments of this invention and are therefore not to be considered limiting of its scope, for the scope of the invention encompasses other equally effective embodiments. The drawings are not necessarily to scale, emphasis generally being placed upon illustrating the features of certain embodiments of the invention. In the drawings, like numerals are used to indicate like parts throughout the various views. Thus, for further understanding of the invention, reference can be made to the following detailed description, read in connection with the drawings in which:

FIG. 1 illustrates a cell scaffold in a protonated and deprotonated state;

FIG. 2 is a flow diagram of a method of forming a cell scaffold;

FIG. 3A shows scaffold swelling as a function of the molar ratio of a pH-responsive monomer to a pH-nonresponsive monomer;

FIG. 3B, shows the mass swelling ratio of a cell scaffold as a function of pH;

FIG. 4 depicts a increase in number of cells increased over time on a pH-responsive cell scaffolds;

FIG. 5 depicts a substrate coiled to form a cylinder;

FIG. 6 depicts a substrate coiled to form a helix;

FIG. 7 is a graph showing control of degree of coiling of a cylinder; and

FIG. 8 is a graph showing control of degree of coiling of a helix.

### DETAILED DESCRIPTION OF THE INVENTION

One of the challenges in tissue engineering is the lack of vascularization of implanted tissue engineered constructs. Cells residing at a critical distance from the blood supply are deprived of oxygen and nutrients which leads to apoptosis. While cell scaffolds have been designed to achieve specific cellular functions (e.g. adhesion, migration, differentiation, etc.) prolonged cell survival has eluded scaffolds that retain a stagnant disposition.

This disclosure provides a cell scaffold that swells in response to a drop in pH, resulting in significant vascularization and cellularizaton compared to non-responsive cell scaffolds. Without wishing to be bound to any particular theory, swelling is believed to increase oxygen transport and, in vivo, induce a pro-healing gene expression profile capable of enhancing angiogenesis. The swelling is reversible such that, when the pH increases, the cell scaffold shrinks.

A cell scaffold that can respond to a cell's environmental milieu may be advantageous in wound healing. A notable change in ischemic tissues is a decrease in pH due primarily to the hydrolysis of ATP and anaerobic glycolysis. An abundance of metabolic waste can also decrease the pH if mass transport properties are not ideal. The disclosed cell scaffold responds to pH cues to swell under such stressed conditions which leads to increased oxygenation and cell survival.

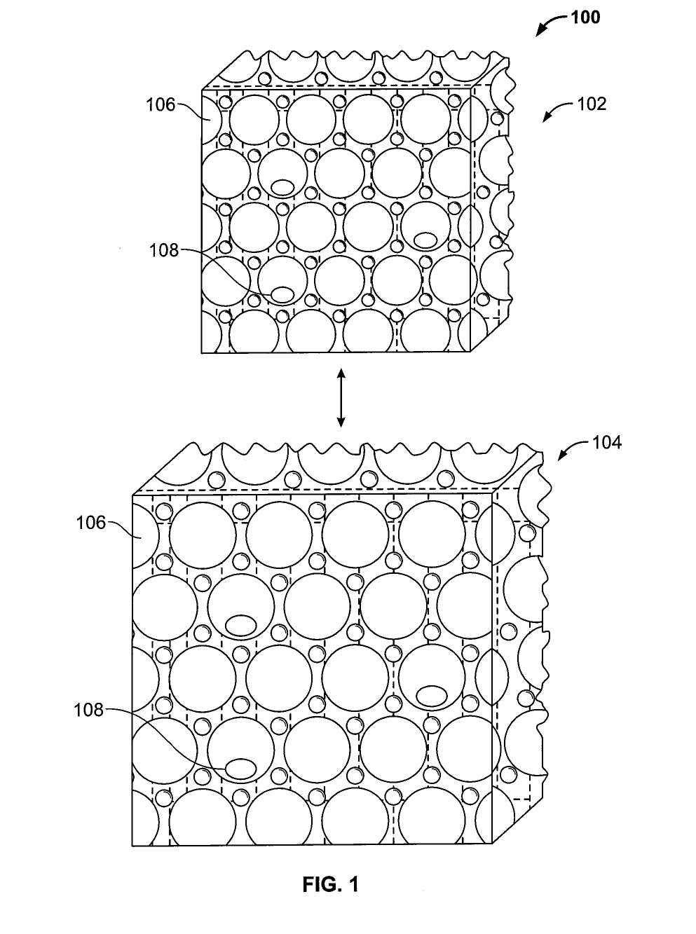

FIG. 1 is a schematic depiction of an example of a cell scaffold 100. The cell scaffold 100 is shown in both an un-protonated state 102 and in a protonated state 104. The cell scaffold 100 comprises a plurality of pores 106 that change in diameter between the un-protonated state 102 and the protonated state 104. A biological cell 108 is disposed in at least some of the pores in the plurality of pores 106.

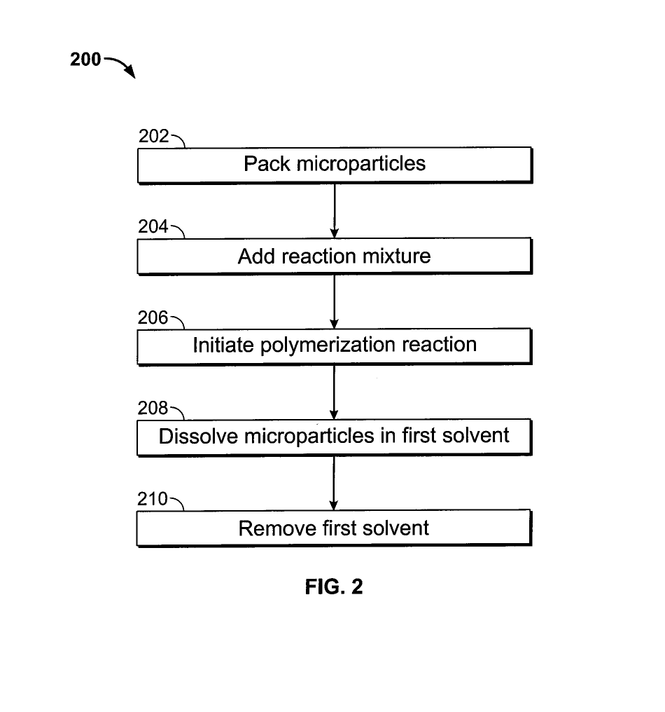

FIG. 2 is a flow diagram of a method 200 of forming a cell scaffold. In step 202, microparticles are packed to provide a lattice. The microparticles are selected to dissolve in a non-aqueous solvent and may be, for example, polymeric microparticles with an average diameter between 100 micrometers and 300 micrometers. In one embodiment, the microparticles are poly(methyl methacrylate) (PMMA) microparticles with an average diameter of about 200 micrometers. In one embodiment, step 202 includes a step of drying the microparticles at an elevated temperature to remove trace water (e.g. drying at 60° C. overnight).

In step 204 of method 200, a reaction mixture is added to the lattice of microparticles. The reaction mixture comprises a pH-nonresponsive monomer and a pH-responsive monomer. Monomers which are pH-responsive are characterized by their formation, after polymerization, of a homopolymer that is protonated or deprotoned within a certain pH range (e.g. a pH of 5-7.5 or, in other embodiments, a pH of 6.5-7.5). In one embodiment, the pH-responsive monomer is an amine-containing monomer. Monomers which are pH-nonresponsive are characterized by their formation, after polymerization, of a homopolymer that is non-responsive to protonation or deprotonation with the same pH range (e.g. a pH of 5-7.5 or, in other embodiments, a pH of 6.5-7.5). For example, the homopolymer formed from the pH-nonresponsive monomer has a pKa outside of the range of 5-7.5 while the homopolymer formed from the pH-responsive monomer has a pKa within the range of 5-7.5.

In the disclosed method, a copolymer is formed from a reaction mixture that comprises both a pH-responsive monomer and a pH-nonresponsive monomer. The resulting copolymer may have a first pKa between about 5 and about 7.5. In one embodiment, the resulting copolymer has a first pKa between about 6.5 and about 7.5. The reaction mixture fills the spaces in the lattice provided by the microparticles. The mole ratio of the pH-nonresponsive monomer to the pH-responsive monomer may be adjusted to tune the sensitivity of the cell scaffold to pH with higher content of the pH-responsive monomer producing more sensitive cell scaffolds. In one embodiment, both monomers are acrylate monomers with the pH-responsive monomer being an amine-containing monomer. For example, the pH-responsive monomer may be N,N-dimethylaminoethyl methacrylate (DMAEMA) and the pH-nonresponsive monomer may be hydroxyethyl methacrylate (HEMA). The resulting copolymer is selected to have a pKa (e.g. between 5-7.5 or, in another embodiment, between 6.5-7.5) suitable for swelling at low pH in a biological environment. In one embodiment, the amine of the amine-containing monomer is a tertiary amine such as an alkyl dimethyl amine or an alkyl diethyl amine. Examples of other suitable monomers are provided in Table 1.

TABLE 1
Examples of monomers

| | pKa | |
|---|---|---|
| monomer | Monomer | Polymer |
| methacrylic acid (MAAc) | pKa = 4.66 | pKa = 5.5 |
| ethylacrylic acid (EAAc) | pKa = 4.97 | pKa = 6.3 |
| propylacrylic acid (PAAc) | pKa = 4. 8 | pKa = 6.7 |
| butylacrylic acid (BAAc) | pKa = 6.8 | pKa = 7.4 |
| maleic acid (MAc) or fumaric acid | Maleic acid has pKa's of | pKa = 5.1-6.6 |
| | 1.83 and 6.07; pKa = 4.4 | |
| | for fumaric acid | |
| itaconic acid | pKa = 4.5 | pKa = 4.9 |
| glutamic acid | pKₐ₁ = 2.2, pKₐ₂ = 4.25, | pKa = 4.9 |
| | pKₐ₃ = 9.67 | |
| Glycerylmonomethacrylate | | |
| (GIMMA) | | |
| Isobutyl methacrylate (IBMA) | | |
| 2-ethylhexyl methacrylate | | |
| (EHMA) | | |
| albumin | pKa = 7.4 | |
| casein | | |
| alginic acid | pKₐ₁ = 3.38, pKₐ₂ = 3.65 | |
| hyaluronic acid | pKa = 3.0 | |
| carrageenan | pKa = 4.9 | |
| Chitosan (polysaccharides) | pKa = 6.0-6.3 | |
| carboxymethyl cellulose | pKa = 4.3 | |
| nucleic acids, such as DNA | | |
| sulfamethazine oligomers | pKa = 7.4 | |
| sulfonamide monomers | pKa = 5.0-7.4 | |
| 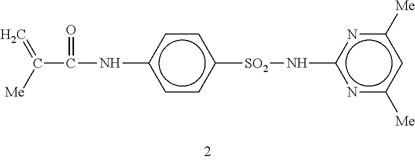 | | |
| N-isopropylacrylamide (NIPAM) | pKa ≈ 6 | pKa = 4.46-10.15 |
| N,N-Dimethylaminoethyl | pKa = 7.3-7.5 | |
| Methacrylate (DMAEMA) | | |
| 2-diethylaminoethyl methacrylate | pKₐ = 7.3 | pKa = 7.0-7.3 |
| (DEAEMA) | | |
| aminoethyl methacrylate (AEMA) | pKa = 8.8 | pKa = 7.6 |
| N-vinyl formamide (NVA) | | pKa = 4-7.4 |
| TEMPO methacrylate | pKa = 5.5 | |
| lysine | pKₐ₁ = 2.2, pKₐ₂ = 9.0; | pKₐ = 10.53 |
| | pKₐ₃ = 10.5 | |
| 2-Methacryloyloxyethyl phosphate | | |
| (MOEP) | | |
| phosphoryl ethyl acrylate (PEA) | | |
| Phosphonic acrylamides | pH = 1.5-2 | |
| 2-acrylamido -2-methyl-1- | | pKa = 6.3 |
| propanesulfonic acid (AMPS) | | |
| 2-methacryloyloxyethyl | pKₐ₁ = 1.9 | |
| phosphorylcholine (MPC) | | |

Examples of phosphonic acrylamides include the following:

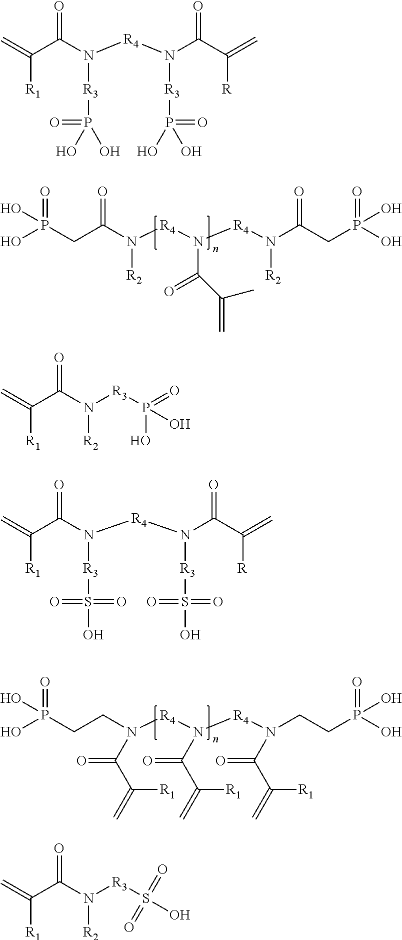

In some embodiments, the reaction mixture further comprises a cross-linking agent. In one embodiment, the cross-linking agent is a bis-acrylate monomer. Examples of suitable cross-linking agents are provided in Table 2.

TABLE 2
Examples of cross-linking agents

| | |
|---|---|
| Methacrylic anhydride | pKa = 4.65 |
| Crotonic anhydride | pKa = 4.61 |
| ethyleneglycoldiacrylate (EGDMA) | pKa = 5.02 (polymer) |
| Tetra(ethylene glycol) diacrylate | |
| triethylene glycol dimethacrylate | |
| (TEGDMA) | |
| Trimethylolpropane trimethacrylate | |
| (TMPTMA) | |
| Methylene bisacrylamide (MBA) | |
| Bis(2-methacryloxyethyl) phosphate | pKₐ₁ = 1.9 |

In step 206, a polymerization reaction is initiated. Depending on the nature of the pH-nonresponsive monomer and the pH-responsive monomer, a variety of initiation processes may be used. In one embodiment, the polymerization reaction is thermally initiated. In another embodiment, the polymerization reaction is photo-initiated by, for example, ultraviolet (UV) light. In some embodiments, rapid polymerization is selected to maintain the microparticles in a fixed position during the polymerization. This facilities formation of the pores 106.

In step 208, the microparticles are dissolved in the non-aqueous solvent and subsequently separated from the copolymer to provide the pores 106. For example, when the microparticles are formed of PMMA, the non-aqueous solvent may be dichloromethane. After the microparticles have been fully dissolved and separated from the copolymer, residual non-aqueous solvent may be removed in step 210 by, for example, prolonged drying, drying under vacuum and/or drying at an elevated temperature. The resulting gel is then washed in water.

In one embodiment, the cell scaffold is formed from a reaction mixture consisting essentially of a mixture of DMAEMA and HEMA and about 3 mol % TEGDMA. Several cellular scaffolds were produced using DMAEMA:HEMA mole ratios of 10/90, 20/80 and 30/70. A control cell scaffold was formed from HEMA and about 3 mol % TEGDMA. As the content of the pH-responsive monomer increased, the resulting cell scaffold became both difficult to handle and increasingly sensitive to pH changes. Cross linkers can be added to facilitate handling, but this decreases the pH sensitivity. A mole ratio of about 30/70 was, for certain applications, found to balance these two properties to an acceptable degree. In one embodiment, the mole ratio of the pH-responsive monomer to the pH-nonresponsive monomer is greater than 20/80 and less than 40/60.

Optical images of the control HEMA and the DMAEMA/HEMA cell scaffolds showed the cell scaffolds taking on a pink hue as DMAEMA content increased. Images of the DMAEMA/HEMA cell scaffolds were taken by bright field microscopy to observe the change in pore size before and after swelling. The pore size of the 30/70 scaffold doubled after swelling at pH 6.5 when compared to the initial non-swollen state at pH 7. Scanning electron micrographs of the control HEMA and the DMAEMA/HEMA cell scaffolds (30/70 mole ratio) demonstrated three-dimensional, interconnected pore structures.

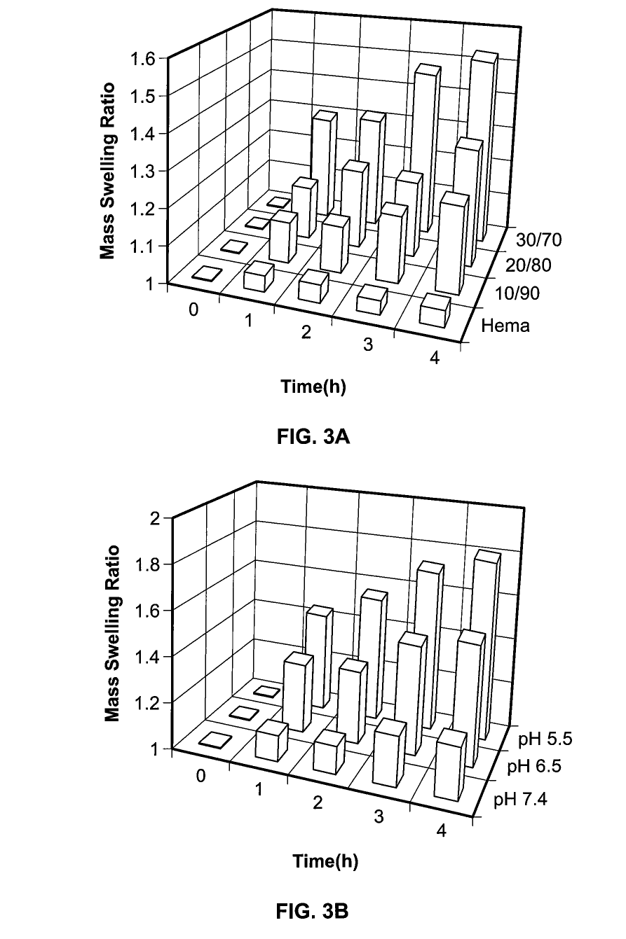

As shown in FIG. 3A, scaffold swelling was measured as a function of the molar ratio of DMAEMA to HEMA. The mass swelling ratio (mass after swell to mass before swell) increased with DMAEMA content. Swelling reflected the density of protonated amine groups in the DMAEMA backbone. This provides the capability of tuning the degree of swelling to a desired extent simply by controlling the molar ratio of DMAEMA to HEMA. As shown in FIG. 3B, the mass swelling ratio of a cell scaffold was also evaluated as a function of pH. As the pH decreased, the swelling ratio of DMAEMA/HEMA scaffolds increased. Without wishing to be bound to any particular theory, water is believed to be convected into DMAEMA/HEMA scaffolds to minimize electrostatic repulsion between cationic amines.

The swelling of the scaffold can alter the elasticity of the cell scaffold by stretching polymer chains. Tensile tests were performed on scaffolds swollen at pH 65 and 7.4 (Instron, Norwood, Mass., USA). The Young's moduli of DMAEMA/HEMA scaffolds were consistently higher than the HEMA scaffold. The Young's moduli of the 10/90 and 30/70 scaffolds at pH 7.4 and pH 6.5 are shown in Table 3. The control HEMA cell scaffold showed no significant change in elasticity at pH 6.5 relative to pH 7.4.

TABLE 3
Young's moduli of selected cell scaffolds

| Mole ratio | Young's moduli (MPa) | Young's moduli (MPa) |
|---|---|---|
| DMAEMA/HEMA | pH 7.4 | pH 6.5 |
| 10/90 | 1.58 ± 0.14 | 1.08 ± 0.12 |
| 30/70 | 2.50 ± 0.39 | 0.39 ± 0.10 |

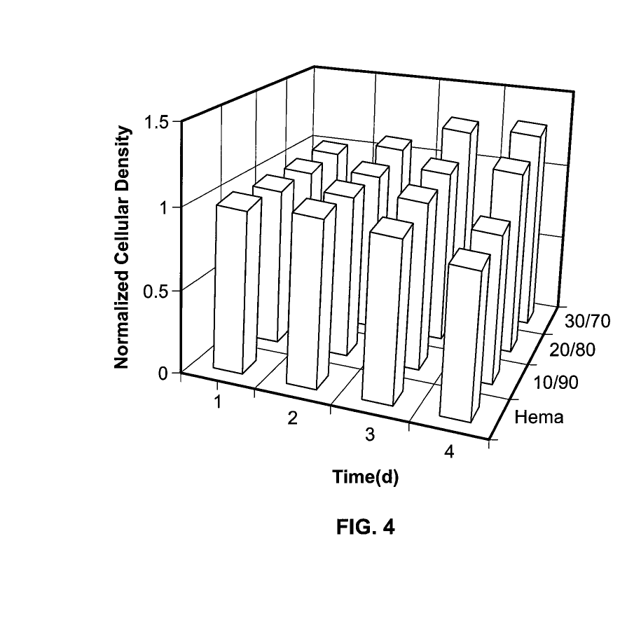

The scaffolds were seeded with mouse embryonic fibroblasts (1.0×10⁶ and 1.0×10⁷ cells per mL) and evaluated for changes in pH and oxygen content (pO₂). The pH of the growth media decreased after 72 h for both cell densities. Similarly, the oxygen level decreased as time progressed over three days. The control HEMA cell scaffold had the lowest pO₂ values and the 10/90 cell scaffold had less oxygen than the 20/80 and 30/70 cell scaffolds. Fluorescence micrographs showed cells homogenous growing throughout the three pH-responsive scaffolds but cells were clustered on the control HEMA scaffold. As shown in FIG. 4, the number of cells increased over the three days on the pH-responsive cell scaffolds. In contrast, the number of cells declined on the control HEMA scaffold over the same period.

### Coiling

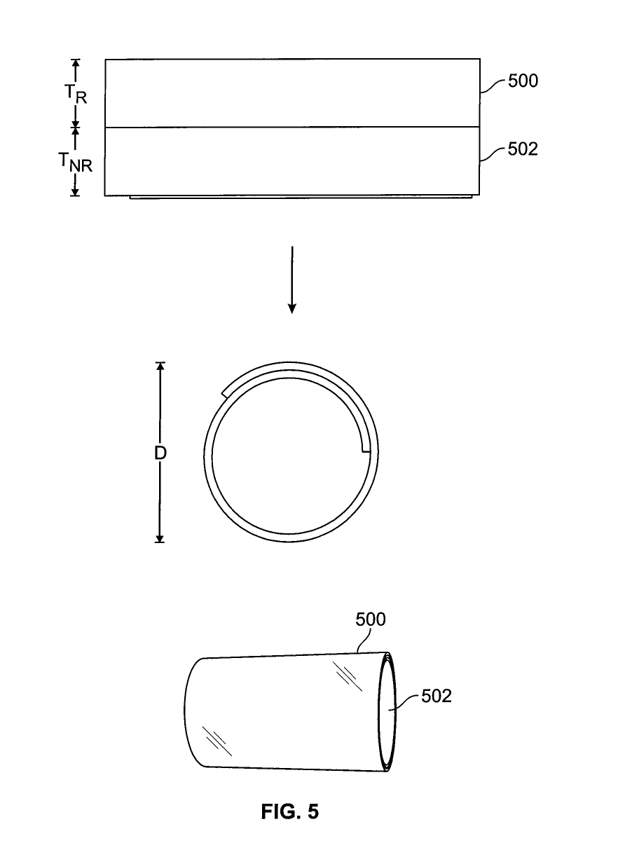

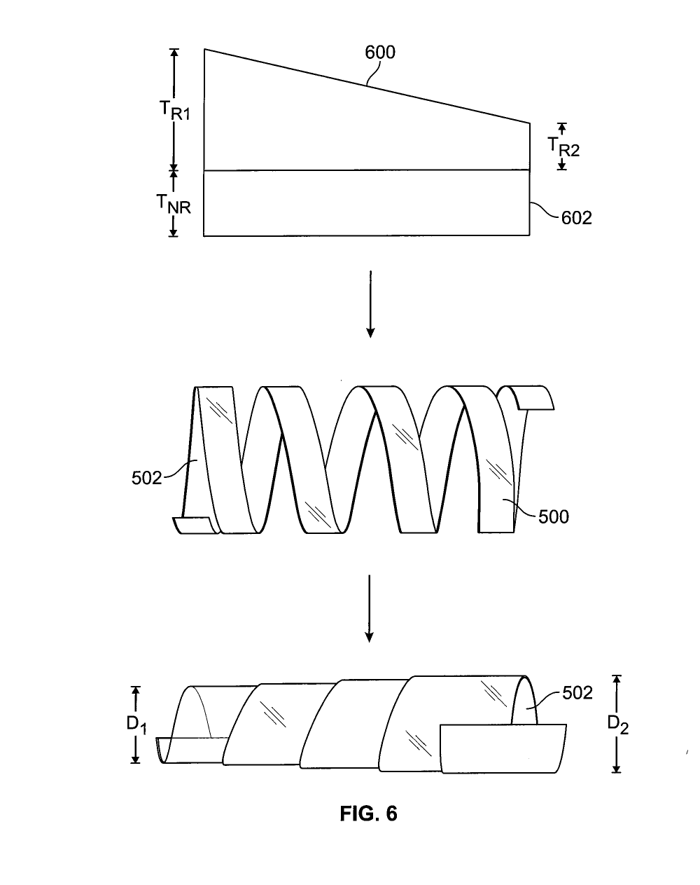

In certain embodiments, a coiled substrate may be produced. The coiled substrate may be a cylinder (FIG. 5) or a helix (FIG. 6). Referring to FIG. 5, a pH-responsive polymer 500 is disposed contiguous with a second polymer 502. The pH-responsive polymer 500 may be formed from a reaction mixture that comprises a pH-nonresponsive monomer and a pH-responsive monomer (e.g. a DMAEMA/HEMA reaction mixture). The second polymer 502 may be formed from a reaction mixture that consists of a single monomer (e.g. a HEMA homopolymer). In another embodiment, the second polymer 502 may be formed from a reaction mixture that comprises a pH-nonresponsive monomer and a pH-responsive monomer (e.g. a DMAEMA/HEMA reaction mixture) but in a different ratio than in pH-responsive polymer 500. The resulting layered structure comprises two polymers, 500, 502, with different degrees of pH sensitivity such that the pH-responsive polymer 500 swells to a greater degree than the second polymer 502. In one embodiment, the pH-responsive polymer 500 is about twice the thickness of the second polymer 502 (e.g. 160 micrometers compared to 80 micrometers. In FIG. 5, the two polymers 500, 502 are of substantially equal thickness. Upon exposure to acidic conditions, the swelling of the pH-responsive polymer 500 produces a curvature that produces a cylinder (FIG. 5). When one of the two polymers, 600, 602 has a thickness that varies over its length, the swelling of the pH-responsive polymer 600 produces a helix (FIG. 6).

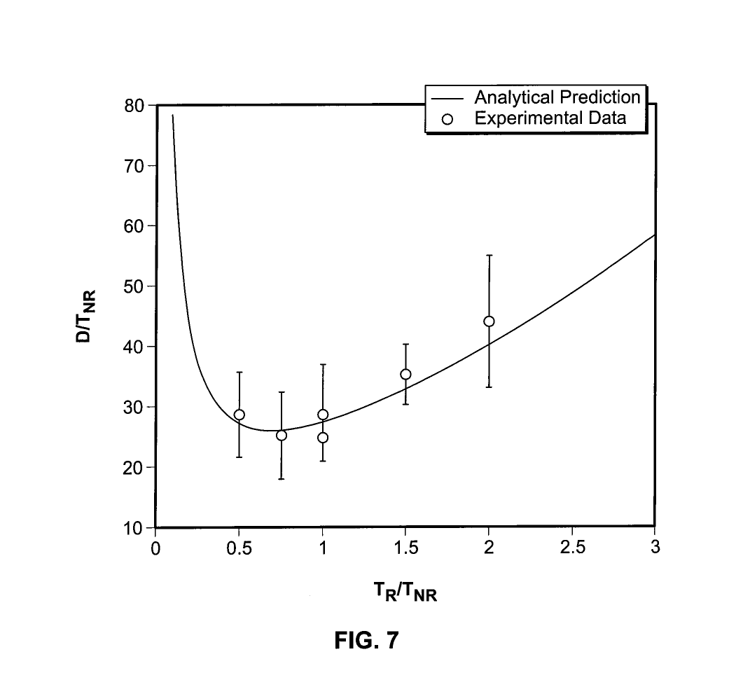

The degree of coiling in a cylinder may be tuned by controlling the relative thickness of the two polymers. Referring to FIG. 5, the thickness (T_{R}) of the pH-responsive polymer 500 and the thickness (T_{NR}) of the second polymer 502 can be adjusted to control the diameter (D) of the resulting coil. As shown in FIG. 7, the ratio of

$$\frac{D}{{T}_{NR}}$$

can be correlated to the ratio of

$$\frac{{T}_{R}}{{T}_{NR}} .$$

demonstrates that experimentally observed values follow with analytically predicted values.

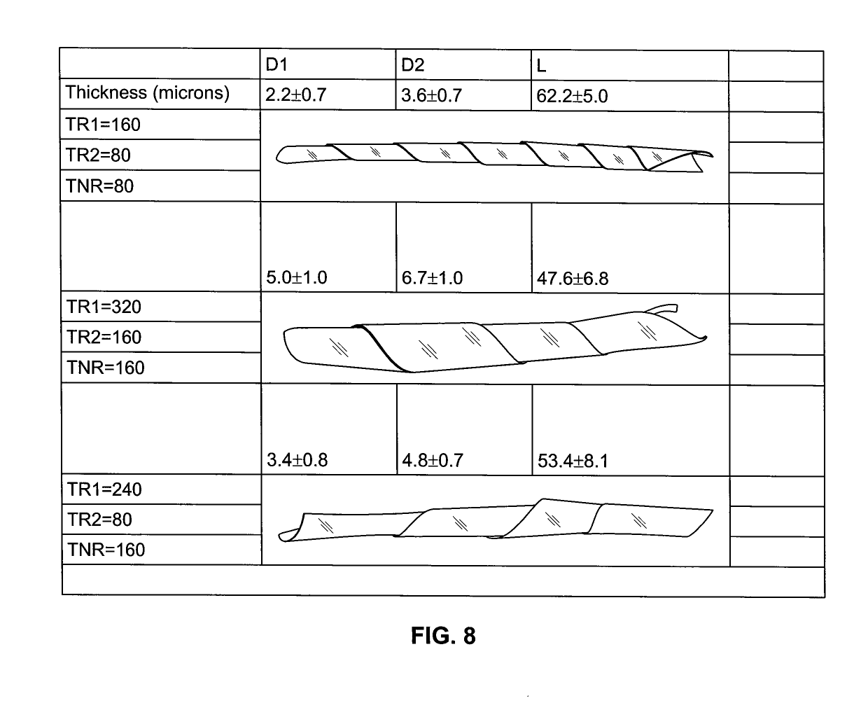

The degree of coiling in a helix may also be tuned by controlling the relative thickness of the two polymers. Referring to FIG. 6, the thickness of the second polymer 602 has a constant thickness (T_{NR}). The pH-responsive polymer 600 has a first thickness (T_{R1}) at a first end of its length (L) and a second thickness (T_{R2}) at a second end of its length. The first thickness (T_{R1}) is greater than the second thickness (T_{R2}). The resulting helix has a first diameter (D₁) at the first end and a second diameter (D₂) at the second end. These diameters and thicknesses are depicted in FIG. 8.

### Modeling of Oxygen Availability

Intracellular processes - biosynthesis, migration and transport—use energy supplied by the coenzyme adenosine-triphosphate (ATP). ATP is the chemical energy depot for molecular and enzymatic reactions and its synthesized in the mitochondria by oxidative phosphorylation. NADPH-linked oxygenase is the enzyme that triggers the respiratory burst that occurs in leukocytes. During the inflammatory phase of wound healing, NADPH-linked oxygenase produces oxidants by consuming high amounts of oxygen, which are needed to prevent infection. Molecular oxygen is also essential for collagen synthesis; hydroxylation of proline and lysine in procollagen is critical to form stable triple helices. Thus, successful wound healing can only occur in the presence of oxygen.

To further evaluate oxygen availability in the cell scaffolds as a function of swelling, a finite difference mathematical model was developed to simulate the oxygen concentration as a function of scaffold width. The scaffolds were assumed to be one-dimensional and taken to be symmetric with respect to x=0. The time-dependent oxygen concentration is equal to the diffusion of oxygen into the cell scaffold minus the rate of consumption:

$$\frac{\partial C ( x , t )}{\partial t} = D \frac{{\partial }^{2} C ( x , t )}{\partial {x}^{2}} - \frac{β {ρ}_{0}}{{λ ( t )}^{2}} \qquad Equation 1$$

where C(x,t) is the oxygen concentration, t is time, D is the diffusion coefficient of oxygen in tissue, x is the distance from the scaffold centerline, β is the oxygen consumption rate per cell, ρ is the cell density and λ(t) is the uniform scaffold stretch. The scaffold stretch λ(t) is described by the half-width of the scaffold x(t) divided by the original half-width x₀, such that λ(t)=x(t)/x₀. This factor is used to describe the change in cell density in equation 1. The swelling of the hydrogel, which was fit to experimental observations, is described by equation 2:

λ(t)=1+(λ_{f}−1)(1−e^{-0.4 t}) Equation 2

where t is in hours. If the initial oxygen concentration is set equal to the external medium, the flux at the center of the scaffold equal to zero due to symmetry, and the oxygen concentration at the periphery constant due to mixing, then the oxygen concentration will decrease at the center of the scaffold as time progresses. In the absence of cell proliferation, the oxygen profile will reach a balance between oxygen diffusion in and oxygen being consumed to provide a necrotic region defined at a distance x from the center of the scaffold. For non-responsive scaffolds (λ₁=1), the necrotic region increased from 0.15 to 0.27 cm in scaffolds that had a fixed half-width of 0.20 and 0.52 cm. In contrast, growth of the necrotic region was halted (at x=0.18) as the 20/70 scaffold expanded from 0.4 to 0.52 cm. Thus, the necrotic region is 67% larger in the non-pH-responsive scaffold relative to the pH-responsive scaffold. The substantial change in the oxygen profile across the 30/70 scaffold is believe to be responsible for the increased cell viability.

### In Vivo Studies

pH-responsive cell scaffolds were implanted subcutaneously in rats for a period of seven to fourteen days to evaluate their performance in vivo. Qualitatively, pH-responsive scaffolds visualized after explantation showed the highest level of cell infiltration. In order to identify cell infiltration and granulation tissue formation, explanted tissue samples were sectioned and strained with hematoxylin and eosin (H&E). A significant increase in granulation tissue was observed after fourteen days in the 30/70 scaffold relative to Sham, the control HEMA scaffold, and the 10/90 cell scaffold.

### Gene Expression Profile Studies

In addition to increased oxygen, cyclic or constant tensile strain was shown to induce cell signal transduction in vitro and in vivo, respectively. Remodeling by integrins and cytoskeletal proteins have been reported to trigger leukocyte recruitment, fibroblast migration and proliferation and angiogenesis. To date, no scaffold has mimicked the pro-healing gene profile characteristic of mechanical manipulation due in part to the body's ability to rapidly equilibrate to stress. The stretch induced by the pH-responsive scaffold may play a role in altering the gene expression profile.

Implanted scaffolds were assessed for vascularization by immunostaining for cluster of differentiation 31 (CD31), which comprises a large portion of endothelial cell intercellular junctions. The 10/90 and 30/70 cell scaffolds exhibited the highest expression of CD31 after seven days of implantation. However, these differences were modest. After fourteen days of implantation, the 10/90 condition showed a marked increase in CD31 expression compared to Sham. Lumen formation was also observed.

Increased cellularization and vascularization in the 30/70 and 10/90 cell scaffold groups indicated a potential therapeutic effect in wound healing. The effect of the cell scaffold implantation of gene expression was therefore evaluated using a Rat Wound Healing PCR Array. The regulation of inflammatory cytokines, growth factors, cell adhesion markers, extracellular matrix (ECM) and signal transduction molecules was examined. The evaluation showed genes were often up-regulated in the 30/70 cell scaffold group relative to Sham and HEMA. A list of all genes analyzed is given in U.S. provisional patent application No. 61/819,180, the content of which is hereby incorporated by reference into this specification. An acute increase in inflammatory cytokines after injury is the first step toward normal wound healing. Cell scaffolds that induce an acute cytokine response to injury may therefore promote would-healing.

In the present study, chemokine (C—X—C motif) ligand 2 (CXCL2) ad Tumor Necrosis Factor alpha (TNFα) were significantly increased in the 30/70 scaffold relative to the control HEMA cell scaffold. CXCL3 regulates angiogenesis via the recruitment and adhesion of leukocytes. The role of TNFα is somewhat controversial in wound healing and depends on concentration, length of exposure and the presence of other cytokines. TNFα is linked to increased collagen production whereas in other reports it is implanted in non-healing wounds. In contrast, CD40 ligand (CD401) was significantly increased in the control HEMA cell scaffold and 10/90 cell scaffold but not in the 30/70 cell scaffold. CD401, a member of the TNF family, leads to inflammation, endothelial dysfunction, neointimal formation after vascular injury and ischemia-reperfusion tissue injury. Significant changes in cytokine regulation that support wound healing were observed in 30/70 scaffolds relative to the control HEMA cell scaffold.

Other cytokines, such as Interleukin-6 (IL6), were up-regulated in pH-responsive scaffolds compared to Sham whereas no statistical significance was observed between the control HEMA cell scaffold and Sham. IL6 is credited with regulating leukocyte infiltration, angiogenesis and collagen accumulation. Knockout and/or antibody blocking of IL6 reduced wound healing in mice.

In the growth factor category, Hepatocytic growth factor (HGF), Heparin binding epidermal growth factor (HBEGF) and VEGF were significantly increased in the 30/70 scaffolds relative to Sham. No statistically significant difference was observed between the control HEMA cell scaffold and Sham. These up-regulated growth factors promote cutaneous wound healing and angiogenesis.

Integrins (Itg) are important cell adhesion molecules that form heterodimers and assist in cell-cell interactions and cell-ECM interactions, thereby participating in all phases of wound healing from immune response to remodeling. Itg α5 was significantly up-regulated in the 30/70 cell scaffold group compared to the control HEMA cell scaffold group. Itg α5 forms heterodimer with β1; α5β1 binds fibronectin via the recognition sequence Arg-Gly-Asp (RGD). α5β1 contributes to keratinocyte migration and angiogenesis. Thus, the 30/70 scaffold may support wound healing by increasing cell adhesion and migration.

Mitogen-activated protein kinase 3 (MAPK3, also known as ERK1), known to play an important role in the VEGF signaling pathway, is significantly increased in the 30/70 cell scaffold group relative to the control HEMA cell scaffold group. MAPK3 regulates cellular proliferation, differentiation, and cell cycle progression in response to cytokines or growth factor stimulation. Of the cytoskeleton proteins, alpha smooth muscle actin (Actin-α2) and actin-β were significantly increased in 30/70 (but not the control HEMA) compared to Sham. This suggests high proliferative and/or synthetic activity of infiltrating smooth muscle and other local cells. Overall, the data suggests that the 20/70 cell scaffold leads to a pro-healing milieu of cytokines and growth factors, which induce signal transduction that results in significant granulocyte tissue formation and vascularization relative to the control HEMA cell scaffold.

### Experimental

### Materials

Dimethylaminoethyl methacrylate (DMAEMA) and 2-hydroxyethyl methacrylate (HEMA) were purchased from Acros (Morris Plains, N.J., USA). Tetraethylene glycol dimethacrylate (TEGDMA) was obtained from Fluka (St. Louis, Mo., USA). Monobasic and dibasic sodium phosphates for the preparation of different pH phosphate buffers and 2,2- dimethoxy-2-phenylacetophoenone (DMPAP) as a photoinitiator were purchased from Sigma (St. Louis, Mo., USA). Dichloromethane was obtained from Mallinckrodt Baker, Inc. (Phillipsburg, N.J., USA). PMMA microparticles with 200 micrometer average diameter (MW 25 k) were purchased from Thermo Fisher Scientific Inc. (Fremont, Calif., USA). All materials were used without further purification. Deionized water (18.2 MΩ) was obtained from a Milli-Q purification system (Millipore Corp., Billerica, Mass., USA).

### Synthesis of pH-Sensitive Scaffolds

PMMA microparticles in diH₂O (20 wt %) were mixed homogeneously and 600 microliters of the microparticle suspension was placed on a Teflon mold (30×10×5 mm). After the PMMA microparticles settled, the water was evaporated at 50° C. in an oven for overnight and then increased temperature up to 140° C. for sintering of PMMA particles for 20 hours. 200 microliters of HEMA and DMAEMA/HEMA solutions (10/90, 20/80, and 30/70, mol/mol) were added in the dried PMMA lattice and then placed under UV light (21.7 mW per square meter, 365 nm) for 90 seconds. After polymerization, the gel was immersed in 20 mL of dichloromethane while shaking vigorously on an orbital shaker for 48 hours to remove the PMMA microparticles. The scaffold was placed in 30 mL of diH₂O and washed for 24 hours three times. Finally, the scaffold was then cut to the desired shape using a cork borer with 8 mm diameter. Also, bright-field microscope (Carl Zeiss, Axiovert 200M, GmbH, Germany) and field emission scanning electron microscopy (FESEM Ultra55, Zeiss, Thornwood, N.Y., USA) were used to characterize the morphologies of the PMMA particles and scaffolds.

### Equilibrium Swelling Studies

Equilibrium swelling of HEMA and DMAEMA/HEMA (10/90, 20/80, and 30/70, mol/mol) scaffolds was performed in a 10 mM sodium phosphate buffered medium of known pH, composition, and temperature. The pH of the buffer was adjusted using 0.1 N HCl to achieve pH 6.5, 7.0, and 7.4. Scaffold strips (30×10×2.0 mm) were placed into a glass jar containing 50 mL of buffer on a shaker (OS-500 orbital shaker, VWR, West Chester, Pa., USA) with a shaking rate of 150±1 rpm in an incubator maintained at 37° C. The swelling ratio of the scaffolds as a function of pH was calculated by measuring the mass of the scaffolds at 0, 1, 2, 3, 4, 24, and 48 h as follows:

$$Swelling Ratio = \frac{{M}_{s}}{{M}_{i}}$$

where Mₛ is the mass of the swollen scaffold in buffer and Mᵢ is the mass of the initial scaffold before swelling.

### Mechanical Testing

The elastic modulus measurement of HEMA and DMAEMA/HEMA (10/90, 20/80, and 30/70, mol/mol) scaffolds were performed in 10 mM phosphate buffers with pH 6.5 and 7.4 after full hydration for three days. Samples were placed on a shaker (150±1 rpm) in an incubator maintained at 37° C. Tensile tests of each scaffold strip (30×10×2.0 mm) were carried out on an Instron BioPuls 5543 (Instron, Norwood, Mass., USA) using a 500 N loading cell. Scaffolds were strained to failure at a rate of 1 mm per min. The Young's modulus was calculated from the initial 40% strain. Five scaffold samples of each formulation were tested.

### Cell Culture

NIH/3T3 mouse fibroblasts (ATCC# CRL-1658) and human umbilical vein endothelial cells (HUVECs, Lonza, Basel, Switzerland) were cultured at 37° C. in humidified air containing 5% carbon dioxide. NIH/3T3 cells were grown in Dulbecco's Modified Eagle's Medium (Invitrogen, Carlsbad, Calif.) supplemented with 10% fetal bovine serum and 1% penicillin and streptomycin, and HUVECs were grown in Endothelial Cell Growth Medium-2 (EGM-2; Lonza, Basel, Switzerland). Cells were seeded into the scaffolds by dropping 100 microliters of cell suspension at either 1.0×10⁶ or 1.0×10⁷ cells per mL onto dry scaffolds. The cells were incubated at 37° C. and 5% carbon dioxide for 2 hr. 1 mL of growth medium was added to each well, and the scaffolds were incubated for 5 days.

### Oxygen Level and pH Change Measurements

Oxygen concentration levels in cell-seeded scaffolds (8×2 mm) were quantified using OxyLab pO₂ pre-calibrated optical fluorescence probes (Oxford Optronix Ltd., Oxford, United Kingdom). HEMA and DMAEMA/HEMA (10/90, 20/80, and 30/70, moVmol) scaffolds were treated with fibronectin (50 micrograms per ml) and incubated at room temperature for one hour in order to allow the extracellular matrix protein to adsorb onto the scaffolds. Scaffolds were seeded with NIH/3T3 fibroblasts and HUVECs and maintained in a humidified incubator at 37° C. and 5% carbon dioxide for two hours in order to allow for cellular attachment within the scaffold to occur. Oxygen levels were quantified at 0, 24, 48, and 72 hour incubation. Measurements were taken in growth media, at the surface and center of the scaffolds for each sample (triplicates for each condition). The oxygen probe was placed at the area of interest until pO₂ readings stabilized. Sensor response time was 5 to 10 seconds, which provided a quasi-real time measurement of the oxygen environment. The pH change of growth medium was measured at different incubation times and cell densities using a pH electrode (PHI 255 pH meter, Beckman Coulter, Brea, Calif., USA).

### Scanning Electron Microscopy

Scaffolds containing cells fixed with 2.5% v/v glutaraldehyde in PBS were dehydrated serially in ethanol, critical point dried with liquid CO2 (Auto Samdri 815 Series A, Tousimis, Rockville, Md.), and coated with Pt/Pd for 2 min with 40 mA with a sputter coater (208HR, Cressington Scientific Instruments, England). Cells were imaged with SEM (FESEM Ultra55, Zeiss, Thomwood, N.Y.) at a beam voltage of 5 kV.

### Simulation Studies

A finite difference mathematical model was developed to predict the oxygen availability in the scaffolds. The 1D model included a scaffold stretching over time increasing its radius from 0.4 cm to 0.52 cm as function of λ(t) (equation 2). These scaffold stretch values were obtained experimentally by performing in vitro swelling studies. Oxygen outside the scaffold was assumed to be constant. Inside the scaffold a diffusion rate D=2×10⁻⁵ cm per s and a consumption rate β=4×10⁻¹⁷ mol/cell·s were modeled according to equation 1. Results were then compared with non-responsive controls where radius was kept constant at 0.4 cm or 0.52 cm. Simulation plots were generated using MATLAB® (The Mathworks, Inc., Natick, Mass.).

### Cell Viability

Cell survival after three and six days was measured using the ALAMARBLUE® fluorometric dye (Invitrogen). 500 microliters of fresh growth medium was added to scaffolds. Scaffolds were incubated for 4 hours at 37° C. after adding a 50 microliters ALAMARBLUE® reagent. Fluorescence was measured at room temperature on a Gemini XPS microplate spectrofluorometer (Molecular Devices, Sunnyvale, Calif.) using an excitation of 570 nm and an emission of 585 nm.

### Animal Surgery

All animal work was approved by the Beth Israel Deaconess Medical Center's (BIDMC), Institutional Animal Care and Use Committee (IACUC). Wistar rats were obtained from Charles River Laboratories (Wilmington, Mass.) and were between 8-12 weeks of age and weighed 350-400 g at the beginning of the study period. 24 Rats were divided into 2 groups for two separate euthanasia time points (7 day or 14 day) post-scaffold implantation.

Rats were anesthetized using Ketamine (40-80 mg/kg IP)+Xylazine (5-10 mg/kg IP). The dorsal trunk was shaved and the skin was prepped for surgery. Four, 1 cm incisions were made on the dorsum of the rat and a subcutaneous pocket was created. Scaffolds (6 mm×2 mm) were then inserted inside the subcutaneous compartment. Each rat received a sham subcutaneous pocket (no scaffold), a 100% HEMA scaffold as a control, a DMAEMA/HEMA (30/70, mol/mol) scaffold, and either a DMAEMA/HEMA (20/80 or 10/90, mol/mol) scaffold. Incisions were sutured (2-3 sutures per incision) with 6-0 nylon monofilament suture and the wounds were covered with a triple antibiotic ointment. After surgery, animals were housed in individual cages.

### Tissue Harvest

On the day of euthanasia, incisions were made around the subcutaneous pockets and 1 cm² sections of skin with the embedded scaffolds were harvested. Each such 1 cm² skin section was divided and processed for histology and immunohistochemistry using paraffin and OCT embedded frozen sections.

### Morphologic Analysis and Immunohistochemistry (IHC)

Tissue samples were fixed in formalin and embedded in paraffin or were embedded in OCT and frozen. For morphologic analysis, 6 micrometer sections were cut and stained with hematoxylin and eosin (H&E). For immunohistochemistry, 6 micrometer sections were cut and deparaffinized in xylene and rehydrated or in case of OCT, sections were fixed with cold acetone. Sections were treated with 3% hydrogen peroxide. Non-serum protein blocking was followed by incubation overnight with primary antibodies (CD31, R&D Systems, Minneapolis, Minn.). Sections were incubated with biotinylated secondary antibodies followed by incubation with the substrate, DAB or Nova Red (Vectastain Mouse Kit; Vectorlabs, Burlingame, Calif.) then counterstained with hematoxylin. To confirm specific staining for each primary antibody, an isotype negative control and a no-primary antibody control was used. All hematoxylin and eosin staining and immunohistochemistry readings were performed in a blinded fashion by two observers and an arbitrary scale of 1-5 was used to grade the extent and intensity of chromogen present. Sections were scored for the extent and intensity of staining: 1: absence of staining; 2: faint scattered staining; 3: moderate staining; 4: intense staining; 5: very intense staining. Protein expression was measured in sham subcutaneous pockets and each of the scaffold embedded skin sections at both euthanasia time points. Qualitative comparison in protein expression is presented as a fold change for the scaffold skin compared to sham skin expression within the same rat. Statistical significance between samples was determined by 2-way ANOVA analysis.

### PCR Array

Tissues from Sham, HEMA, 10/90, and 30/70 samples were snap frozen in liquid nitrogen and stored at −80° C. until processing. Total RNA was isolated using the RNease Microarray Tissue kit (Qiagen) according to the manufacturer's protocol. Yield and purity were quantified using a SpectraMax Plus 384 spectrophotometer (Molecular Devices). cDNA was prepared using 1 microgram RNA with the RT² First Strand kit (Qiagen). Real-time PCR (RT-PCR) was performed on a Rat Wound Healing RT² Profiler PCR Array (SABiosciences). RT² Profiler PCR Array Data Analysis version 3.5 (SABiosciences) was used to evaluate gene expression profiles. Ribosomal protein L13A (Rp113a) and Lactase dehydrogenase A (Ldna) were used as housekeeping genes. Statistical significance was determined by unpaired Student's t-test with p<0.05.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A pH-responsive cell scaffold comprising:
a polymeric scaffold formed from a polymerization reaction of a reaction mixture comprising a pH-nonresponsive monomer and a pH-responsive monomer, the polymerization reaction forming a copolymer with a first pKa between about 5 and about 7.5; and
a plurality of pores in the polymeric scaffold, wherein the polymeric scaffold swells when exposed to an environment with a pH below the first pKa.

2. The cell scaffold as recited in claim 1, wherein the pH-nonresponsive monomer is characterized by its corresponding homopolymer having a pKa outside of a range of about 5 and about 7.5 while the pH-responsive monomer is characterized by its corresponding homopolymer having a pKa inside of the range of about 5 and about 7.5.

3. The cell scaffold as recited in claim 1, wherein the pH-responsive monomer has a tertiary amine.

4. The cell scaffold as recited in claim 1, wherein the first pKa is between about 6.5 and about 7.5.

5. The cell scaffold as recited in claim 4, wherein the pH-nonresponsive monomer is characterized by its corresponding homopolymer having a pKa outside of a range of about 6.5 and about 7.5 while the pH-responsive monomer is characterized by its corresponding homopolymer having a pKa inside of the range of about 6.5 and about 7.5.

6. The cell scaffold as recited in claim 1, further comprising a plurality of biological cells disposed in at least some of the pores of the plurality of pores.

7. The cell scaffold as recited in claim 1, wherein the pH-responsive monomer and the pH-nonresponsive monomer are both acrylate monomers.

8. The cell scaffold as recited in claim 1, wherein the pH-nonresponsive monomer is 2-hydroxyethyl methacrylate (HEMA).

9. The cell scaffold as recited in claim 1, wherein the pH-responsive monomer is dimethylaminoethyl methacrylate (DMAEMA).

10. The cell scaffold as recited in claim 1, wherein the pH-nonresponsive monomer is 2-hydroxyethyl methacrylate (HEMA) and the pH-responsive monomer is dimethylaminoethyl methacrylate (DMAEMA).

11. The cell scaffold as recited in claim 1, wherein the pH-responsive monomer and the pH-nonresponsive monomer are present in a mole ratio of at least about 20 to 80 and less than 40 to 80.

12. The cell scaffold as recited in claim 10, wherein the pH-responsive monomer and the pH-nonresponsive monomer are present in a mole ratio of at least about 30 to 70.

13. The cell scaffold as recited in claim 1, wherein the pores in the plurality of pores have an average diameter between about 100 micrometers and 300 micrometers.

14. The cell scaffold as recited in claim 1, wherein the pores in the plurality of pores are uniformly distributed throughout the polymeric scaffold.

15. The cell scaffold as recited in claim 1, wherein the reaction mixture further comprises a cross-linking agent.

16. The cell scaffold as recited in claim 15, wherein the cross-linking agent is a bis-acrylate.

17. A method of growing a cell culture, the method comprising steps of:
permitting biological cells to be disposed in a plurality of pores in a polymeric scaffold, the polymeric scaffold being formed from a polymerization reaction of a reaction mixture comprising a pH-nonresponsive monomer and a pH-responsive monomer, the polymerization reaction forming a copolymer with a first pKa between about 5 and about 7.5, wherein the polymeric scaffold swells when exposed to an environment with a pH below the first pKa; and
allowing the biological cells to adsorb nutrients from an ambient environment and grow a cell culture.

18. The method as recited in claim 17, further comprising placing the polymeric scaffold in contact with biological tissue, wherein the step of permitting biological cells to be disposed in the plurality of pores permits biological cells from the biological tissue to enter the plurality of pores.

19. The method as recited in claim 18, wherein the biological tissue is part of a biological organism such that the method is performed in vivo.

20. A coiled substrate comprising
a first polymer formed from a polymerization reaction of a reaction mixture comprising a pH-nonresponsive monomer and a pH-responsive monomer, the polymerization reaction forming a copolymer with a first pKa between about 5 and about 7.5, the copolymer having a first degree of pH-responsive swelling;
a second polymer, contiguous with the first polymer, the second polymer having a second degree of pH-responsive swelling, different than the first degree of pH-responsive swelling.
